SEQUENCE LISTING (10 sequences)

SEQ ID NO: 1 - DNA, 1176 bp, Saccharomyces sp.
atgtctgctgctgctgatagattaaacttaacctctggccatttgaacgccggtagaaagagaagttcttcttctgtttctttgaaggccgctgaaaagcctttcaaggtcaccgtcattggttccggtaactggggtaccaccattgcaaaggtcgttgctgaaaactgtaagggttacccagaggttttcgctccagaagttcaaatgtgggtgtttgaagaagacattaacggtgaaaaacttaccgaaatcataaataccagacaccaaaacgtcaagtacctgccaaatatcactcttccagacaacctggtcgccaacccagatttgattgactccgttaaggacgtcgacattctagtgttcaacatcccacatcaatttttgccacgtatctgtggccaattgaagggtcacgtcagctctcacgtcagagctatctcctgtttgaagggtttcgaagtcggtgccaagggtgtccaattgttgtcctcttacatcaccgaaaacctaggcatccaatgtggtgctttatccggtgctaacattgccacagaggtcgcccaagaacattggtctgaaaccacagttgcctaccacattccaaaggactttagaggtgagggtaaggatgtcgaccacaaggtcttgaaagccttgttccacagaccttacttccacgtcagtgtcattgaagatgtcgccggtatctccatctgtggtgctttgaagaacgtcgtcgccttgggttgcggtttcgtcgaaggtctgggctggggtaacaacgcttctgccgccattcaaagagtcggtctgggtgagatcataaggttcggccaaatgtttttcccagaatctagagaagaaacctactatcaagaatccgctggtgttgctgacttgatcaccacctgtgccggtggtagaaacgtcaaggttgctagattaatggctacttctggtaaggatgcctgggagtgtgaaaaagagttgttgaacggccaatccgctcaaggtttgatcacctgtaaggaagtccacgaatggttagaaacctgtgggtccgttgaagatttcccattgttcgaagccgtctaccaaatcgtttacaacaactacccaatgaagaacctgccggatatgattgaagaattagatctacacggagaatag

SEQ ID NO: 2 - protein, 391 aa, Saccharomyces sp.
MSAAADRLNLTSGHLNAGRKRSSSSVSLKAAEKPFKVTVIGSGNWGTTIAKVVAENCKGYPEVFAPEVQMWVFEEDINGEKLTEIINTRHQNVKYLPNITLPDNLVANPDLIDSVKDVDILVFNIPHQFLPRICGQLKGHVSSHVRAISCLKGFEVGAKGVQLLSSYITENLGIQCGALSGANIATEVAQEHWSETTVAYHIPKDFRGEGKDVDHKVLKALFHRPYFHVSVIEDVAGISICGALKNVVALGCGFVEGLGWGNNASAAIQRVGLGEIIRFGQMFFPESREETYYQESAGVADLITTCAGGRNVKVARLMATSGKDAWECEKELLNGQSAQGLITCKEVHEWLETCGSVEDFPLFEAVYQIVYNNYPMKNLPDMIEELDLHGE

SEQ ID NO: 3 - DNA, 1320 bp, Saccharomyces sp.
atgcttgctctcagacggttaacaagatacacattcctcaagcgaacgcatccggtgttatctgctcatcgtgcatataaatttttccctttaagttccacttgctcaagaagaacatttcagatacaactgcgctcaaagatgactgctcatatcaaccacaaatcgcacaacggtaaccaagatgaccatccgttcaaaagatcagactctgcggtgtcgattatgcatttgaaacgtgagcctttcaagatcacagtgattggttccggtaactgggggaccaccatcgccaaagttattgcagaaaacacagagctgcattctcatattttcaacccggaagtgagaatgtgggtgtttgatgagaaaatcggtgacgaaaatctaacggatatcataaatacaagacatcaaaacgtcaaatatctaccaaatatcgacctgccccacaacttggtggctgatcccgatcttttgcattccatcaagggtgctgatatccttgtcttcaacatccctcatcaattcttacctaatatagtcaaacagctacaaggccacgtggcccctcacgtaagagccatctcgtgtctcaaagggttcgaactgggctccaagggtgttcagttgctgtcctcctacgtcaccgacgagttaggagtccaatgtggtgcattatccggcgccaacctagcccccgaagtggccaaggaacactggtccgaaaccaccgtggcctaccaactgccaaaggactaccaaggtgaaggcaaggacgtagaccacaaggttctaaaactactgttccacagaccttacttccacgtcaacgtcatcgacgatgttgctggtatctccattgcaggtgctttgaaaaacgtcgtagcactggcctgcgggttcgtagaaggcatgggatggggtaacaacgcctccgctgccatccagagactgggtctgggcgagatcatcaagttcgggagaatgttcttcccggaatccaaggtcgagacctactaccaggagtccgctggtgtggctgatctaatcaccacatgttcaggcggcagaaacgtcaaggtcgctacgtacatggccaagaccggcaagtccgccctggaagcagaaaaggaactgctaaacggccagtccgcccaggggatcatcacatgcagagaagtccacgagtggctgcaaacgtgtgagttgacccaagaatacccactgttcgaggcggtgtaccagatagtttacaacaacgtccgcatggaagacctaccggaaatgattgaagaactagacatcgaggacaaatag

SEQ ID NO: 4 - protein, 439 aa, Saccharomyces sp.
MLALRRLTRYTFLKRTHPVLSAHRAYKFFPLSSTCSRRTFQIQLRSKMTAHINHKSHNGNQDDHPFKRSDSAVSIMHLKREPFKITVIGSGNWGTTIAKVIAENTELHSHIFNPEVRMWVFDEKIGDENLTDIINTRHQNVKYLPNIDLPHNLVADPDLLHSIKGADILVFNIPHQFLPNIVKQLQGHVAPHVRAISCLKGFELGSKGVQLLSSYVTDELGVQCGALSGANLAPEVAKEHWSETTVAYQLPKDYQGEGKDVDHKVLKLLFHRPYFHVNVIDDVAGISIAGALKNVVALACGFVEGMGWGNNASAAIQRLGLGEIIKFGRMFFPESKVETYYQESAGVADLITTCSGGRNVKVATYMAKTGKSALEAEKELLNGQSAQGIITCREVHEWLQTCELTQEYPLFEAVYQIVYNNVRMEDLPEMIEELDIEDK

SEQ ID NO: 5 - DNA, 40 bp, Artificial Sequence
  Primer
gagctcatagcggccatgtctgctgctgctgatagattaa

SEQ ID NO: 6 - DNA, 42 bp, Artificial Sequence
  Primer
ggatcctatgcggccgcgttatgggaaatgacataatgctag

SEQ ID NO: 7 - DNA, 40 bp, Artificial Sequence
  Primer
gagctcatagcggccatgcttgctctcagacggttaacaa

SEQ ID NO: 8 - DNA, 42 bp, Artificial Sequence
  Primer
ggatcctatgcggccgcagtagtagtagtagtagtactagag

SEQ ID NO: 9 - DNA, 1176 bp, Saccharomyces sp.
atgtctgctgctgctgatagattaaacttaacttccggccacttgaatgctggtagaaagagaagttcctcttctgtttctttgaaggctgccgaaaagcctttcaaggttactgtgattggatctggtaactggggtactactattgccaaggtggttgccgaaaattgtaagggatacccagaagttttcgctccaatagtacaaatgtgggtgttcgaagaagagatcaatggtgaaaaattgactgaaatcataaatactagacatcaaaacgtgaaatacttgcctggcatcactctacccgacaatttggttgctaatccagacttgattgattcagtcaaggatgtcgacatcatcgttttcaacattccacatcaatttttgccccgtatctgtagccaattgaaaggtcatgttgattcacacgtcagagctatctcctgtctaaagggttttgaagttggtgctaaaggtgtccaattgctatcctcttacatcactgaggaactaggtattcaatgtggtgctctatctggtgctaacattgccaccgaagtcgctcaagaacactggtctgaaacaacagttgcttaccacattccaaaggatttcagaggcgagggcaaggacgtcgaccataaggttctaaaggccttgttccacagaccttacttccacgttagtgtcatcgaagatgttgctggtatctccatctgtggtgctttgaagaacgttgttgccttaggttgtggtttcgtcgaaggtctaggctggggtaacaacgcttctgctgccatccaaagagtcggtttgggtgagatcatcagattcggtcaaatgtttttcccagaatctagagaagaaacatactaccaagagtctgctggtgttgctgatttgatcaccacctgcgctggtggtagaaacgtcaaggttgctaggctaatggctacttctggtaaggacgcctgggaatgtgaaaaggagttgttgaatggccaatccgctcaaggtttaattacctgcaaagaagttcacgaatggttggaaacatgtggctctgtcgaagacttcccattatttgaagccgtataccaaatcgtttacaacaactacccaatgaagaacctgccggacatgattgaagaattagatctacatgaagattag

SEQ ID NO: 10 - protein, 391 aa, Saccharomyces sp.
MSAAADRLNLTSGHLNAGRKRSSSSVSLKAAEKPFKVTVIGSGNWGTTIAKVVAENCKGYPEVFAPIVQMWVFEEEINGEKLTEIINTRHQNVKYLPGITLPDNLVANPDLIDSVKDVDIIVFNIPHQFLPRICSQLKGHVDSHVRAISCLKGFEVGAKGVQLLSSYITEELGIQCGALSGANIATEVAQEHWSETTVAYHIPKDFRGEGKDVDHKVLKALFHRPYFHVSVIEDVAGISICGALKNVVALGCGFVEGLGWGNNASAAIQRVGLGEIIRFGQMFFPESREETYYQESAGVADLITTCAGGRNVKVARLMATSGKDAWECEKELLNGQSAQGLITCKEVHEWLETCGSVEDFPLFEAVYQIVYNNYPMKNLPDMIEELDLHED

(19) United States
(12) Patent Application Publication
(10) Pub. No.: US 2009/0004330 A1
(43) Pub. Date: Jan. 1, 2009

(54) Glycerol-3-Phosphate Dehydrogenase Gene And Use Thereof

(75) Inventors: Yoshihiro Nakao, Osaka (JP); Yukiko Kodama, Osaka (JP); Tomoko Shimonaga, Osaka (JP)

Correspondence Address: DRINKER BIDDLE & REATH (DC), 1500 K STREET, N.W., SUITE 1100, WASHINGTON, DC, 20005-1209, US

(73) Assignee: SUNTORY LIMITED, OSAKA (JP)

(21) Appl. No.: 12/280,725

(22) PCT Filed: Feb. 21, 2007
(86) PCT No.: PCT/JP2007/053704

§ 371 Date: Aug. 26, 2008

(30) Foreign Application Priority Data
Mar. 1, 2006 (JP) 2006-055556

(51) Int. Cl. C12G 1/00; C12N 15/11; C12N 9/02; C12N 15/00; C12N 1/19; C12Q 1/68; C12Q 1/32; C12N 15/87; C12C 1/00

(52) U.S. Cl. 426/16; 536/23.2; 435/189; 435/320.1; 435/254.2; 435/6; 435/26; 435/471; 426/11

(57) ABSTRACT

The present invention relates to a gene encoding glycerol-3-phosphate dehydrogenase and use thereof, in particular, a brewer's yeast which produces alcoholic beverages with superior body and mellowness, alcoholic beverages produced with said yeast, and a method for producing said beverages. More particularly, the present invention relates to a yeast whose ability of producing glycerol, which contribute to body and mellowness of products, is enhanced by amplifying expression level of GPD1 or GPD2 gene encoding a Gpd1p or Gpd2p which is a glycerol-3-phosphate dehydrogenase in brewer's yeast, especially non-ScGPD1 gene or non-ScGPD2 gene specific to a lager brewing yeast and to a method for producing alcoholic beverages with said yeast, etc.

### TECHNICAL FIELD

The present invention relates to a glycerol-3-phosphate dehydrogenase gene and use thereof, in particular, a brewer's yeast which produces alcoholic beverages with superior body and mellowness, a brewery yeast with superior low-temperature storage property, frozen storage property, drying resistant property or osmotic-pressure resistant property, alcoholic beverages produced with said yeast, and a method for producing said beverages. More particularly, the present invention relates to a yeast which is able to enhance body and mellowness of a product by amplifying expression level of GPD1 or GPD2 gene encoding Gpd1p or Gpd2p which is a glycerol-3-phosphate dehydrogenase in brewer's yeast, especially non-ScGPD1 gene or non-ScGPD2 gene specific to a lager brewing yeast, a yeast with superior low-temperature storage property, frozen storage property, drying resistant property or osmotic-pressure resistant property and to a method for producing alcoholic beverages with said yeast, etc.

### BACKGROUND ART

Glycerol, which is said to contribute to body and mellowness as well as sweetness, is one of the important taste components of alcoholic beverages.

Glycerol high-producing yeasts have been developed to increase glycerol levels in alcoholic beverages. A method employing resistant property to allyl alcohol or pyrazole as an indicator (Japanese Examined Patent Publication (Kokoku) No. H7-89901), a method employing resistant property to glycerol monochlorohydrin as an indicator (Japanese Patent Application Laid-open No. H10-210968), and a method employing resistant property to salts as an indicator (Japanese Patent Application Laid-open No. H7-115956), a method employing resistant property to amino acid analogues as a indicator (J. Ferment. Bioeng., 80, 218-222 (1995)) for mutating yeast and isolating glycerol high-producing yeasts effectively have been reported.

On the other hand, a method in which a Glycerol-3-phosphate dehydrogenase GPD1 or GPD2 was highly expressed in beer yeasts or wine yeasts was reported as a method employing development of yeast by gene manipulation technology (FEMS Yeast Res. 2: 225-232 (2002), Appl. Environ. Microbiol. 65: 143-149 (1999), Austr. J. Grape Wine Res. 6: 208-215 (2000)).

Further, yeast is known to synthesize and accumulate glycerol, which is an osmolyte, to cancel osmotic pressure difference between inside and outside of the cell when it is exposed to high osmotic stress. Genes of glycerol production pathway including glycerol-3-phosphate dehydrogenase are known to be induced by stress (Microbiol Mol Biol Rev. 66: 300-372 (2002)).

### DISCLOSURE OF INVENTION

As noted above, variant strains have been developed in order to increase glycerol level in a product. As a result, however, unexpected delays in fermentation and increases in undesirable flavor components have been observed in some cases, which make the practical use of such yeast questionable. There were thus demands for a method of developing yeast which can produce sufficient glycerol without compromising either fermentation rate or product quality.

The present inventors made extensive studies to solve the above problems and as a result, succeeded in identifying and isolating a gene encoding glycerol-3-phosphate dehydrogenase from beer yeast. Moreover, the present inventors produced transformed yeast in which the obtained gene was expressed to verify that the production amount of glycerol can be actually elevated, thereby completing the present invention.

Thus, the present invention relates to a glycerol-3-phosphate dehydrogenase gene of lager brewing yeast, to a protein encoded by said gene, to a transformed yeast in which the expression of said gene is controlled, to a method for controlling production amount of glycerol in products using a yeast in which the expression of said gene is controlled, or the like. More specifically, the present invention provides the following polynucleotides, a vector comprising said polynucleotide, a transformed yeast introduced with said vector, a method for producing alcoholic beverages by using said transformed yeast, and the like.

(1) A polynucleotide selected from the group consisting of:

(a) a polynucleotide comprising a polynucleotide consisting of the nucleotide sequence of SEQ ID NO: 1 or SEQ ID NO: 3;

(b) a polynucleotide comprising a polynucleotide encoding a protein consisting of the amino acid sequence of SEQ ID NO: 2 or SEQ ID NO: 4;

(c) a polynucleotide comprising a polynucleotide encoding a protein consisting of the amino acid sequence of SEQ ID NO: 2 or SEQ ID NO: 4 in which one or more amino acids thereof are deleted, substituted, inserted and/or added, and having a glycerol-3-phosphate dehydrogenase activity;

(d) a polynucleotide comprising a polynucleotide encoding a protein having an amino acid sequence having 60% or higher identity with the amino acid sequence of SEQ ID NO: 2 or SEQ ID NO: 4, and said protein having a glycerol-3-phosphate dehydrogenase activity;

(e) a polynucleotide comprising a polynucleotide which hybridizes to a polynucleotide consisting of a nucleotide sequence complementary to the nucleotide sequence of SEQ ID NO: 1 or SEQ ID NO: 3 under stringent conditions, and which encodes a protein having a glycerol-3-phosphate dehydrogenase activity; and

(f) a polynucleotide comprising a polynucleotide which hybridizes to a polynucleotide consisting of a nucleotide sequence complementary to the nucleotide sequence of the polynucleotide encoding the protein having the amino acid sequence of SEQ ID NO: 2 or SEQ ID NO: 4 under stringent conditions, and which encodes a protein having a glycerol-3-phosphate dehydrogenase activity.

(2) The polynucleotide according to (1) above selected from the group consisting of:

(g) a polynucleotide comprising a polynucleotide encoding a protein consisting of the amino acid sequence of SEQ ID NO: 2 or SEQ ID NO: 4, or encoding the amino acid sequence of SEQ ID NO: 2 or SEQ ID NO: 4 in which 1 to 10 amino acids thereof are deleted, substituted, inserted, and/or added, and wherein said protein has a glycerol-3-phosphate dehydrogenase activity;

(h) a polynucleotide comprising a polynucleotide encoding a protein having 90% or higher identity with the amino acid sequence of SEQ ID NO: 2 or SEQ ID NO: 4, and having a glycerol-3-phosphate dehydrogenase activity; and

(i) a polynucleotide comprising a polynucleotide which hybridizes to a polynucleotide consisting of a nucleotide sequence of SEQ ID NO: 1 or SEQ ID NO: 3 or which hybridizes to a polynucleotide consisting of a nucleotide sequence complementary to the nucleotide sequence of SEQ ID NO: 1 or SEQ ID NO: 3, under high stringent conditions, which encodes a protein having a glycerol-3-phosphate dehydrogenase activity.

(3) The polynucleotide according to (1) above comprising a polynucleotide consisting of the nucleotide sequence of SEQ ID NO: 1 or SEQ ID NO: 3.

(4) The polynucleotide according to (1) above comprising a polynucleotide encoding a protein consisting of the amino acid sequence of SEQ ID NO: 2 or SEQ ID NO: 4.

(5) The polynucleotide according to any one of (1) to (4) above, wherein the polynucleotide is DNA.

(6) A protein encoded by the polynucleotide according to any one of (1) to (5) above.

(7) A vector containing the polynucleotide according to any one of (1) to (5) above.

(7a) The vector of (7) above, which comprises the expression cassette comprising the following components:

(x) a promoter that can be transcribed in a yeast cell;

(y) any of the polynucleotides described in (1) to (5) above linked to the promoter in a sense or antisense direction; and

(z) a signal that can function in a yeast with respect to transcription termination and polyadenylation of a RNA molecule.

(8) A yeast into which the vector according to (7) or (7a) above has been introduced.

(9) The yeast according to (8) above, wherein glycerol producing ability is increased.

(9a) A yeast whose glycerol producing ability is increased by introducing the vector of (8) above.

(10) The yeast according to any one of (8) to (9a) above, wherein low-temperature storage property, frozen storage property or drying-resistant property is increased.

(11) The yeast according to any one of (8) to (9a) above, wherein osmotic pressure resistant property is increased.

(12) The yeast according to (9) or (9a) above, wherein the glycerol producing ability is increased by increasing an expression level of the protein of (6) above.

(13) The yeast according to (10) above, wherein the low-temperature storage property, frozen storage property or drying-resistant property is increased by increasing an expression level of the protein of (6) above.

(14) The yeast according to (11) above, wherein the osmotic pressure resistant property is increased by increasing an expression level of the protein of (6) above.

(15) A method for producing an alcoholic beverage by using the yeast according to any one of (8) to (14) above.

(16) The method according to (15) above, wherein the brewed alcoholic beverage is a malt beverage.

(17) The method according to (15) above, wherein the brewed alcoholic beverage is wine.

(18) An alcoholic beverage produced by the method according to any one of (15) to (17) above.

(19) A method for assessing a test yeast for its glycerol producing ability, comprising: using a primer or probe designed based on the nucleotide sequence of a glycerol-3-phosphate dehydrogenase gene having the nucleotide sequence of SEQ ID NO: 1 or SEQ ID NO: 3.

(19a) A method for selecting a yeast having a high glycerol producing ability by using the method described in (19) above.

(19b) A method for producing an alcoholic beverage (for example, beer) by using the yeast selected with the method described in (19a) above.

(20) A method for assessing a test yeast for its glycerol producing ability, comprising: culturing the test yeast; and measuring the expression level of the glycerol-3-phosphate dehydrogenase gene having the nucleotide sequence of SEQ 11D NO: 1 or SEQ ID NO: 3.

(20a) A method for selecting a yeast having a high glycerol producing ability, which comprises assessing a test yeast by the method described in (20) above and selecting a yeast having a high expression level of the glycerol-3-phosphate dehydrogenase gene.

(20b) A method for producing an alcoholic beverage (for example, beer) by using the yeast selected with the method described in (20a) above.

(21) A method for selecting a yeast, comprising: culturing test yeasts; quantifying the protein of (6) above or measuring the expression level of the glycerol-3-phosphate dehydrogenase gene having the nucleotide sequence of SEQ ID NO: 1 or SEQ ID NO: 3; and selecting a test yeast having an amount of the protein or the gene expression level according to desired glycerol producing ability.

(21a) A method for selecting a yeast, comprising: culturing test yeasts; measuring glycerol producing ability or glycerol-3-phosphate dehydrogenase activity; and selecting a test yeast having a desired glycerol producing ability or glycerol-3-phosphate dehydrogenase activity.

(22) The method for selecting a yeast according to (21) above, comprising: culturing a reference yeast and test yeasts; measuring for each yeast the expression level of the glycerol-3-phosphate dehydrogenase gene having the nucleotide sequence of SEQ ID NO: 1 or SEQ ID NO: 3; and selecting a test yeast having the gene expression higher than that in the reference yeast.

(23) The method for selecting a yeast according to (21) above, comprising: culturing a reference yeast and test yeasts; quantifying the protein according to (6) above in each yeast; and selecting a test yeast having a larger amount of the protein than that in the reference yeast.

(24) A method for producing an alcoholic beverage comprising: conducting fermentation using the yeast according to any one of (8) to (14) above or a yeast selected by the methods according to any one of (21) to (23) above; and controlling production amount of glycerol.

(25) A method for enhancing low-temperature storage property of yeast by using a vector comprising a polynucleotide selected from the group consisting of

(j) a polynucleotide comprising a polynucleotide encoding a protein consisting of the amino acid sequence of SEQ ID NO: 10, or encoding the amino acid sequence of SEQ ID NO: 10 in which 1 to 10 amino acids thereof are deleted, substituted, inserted, and/or added, and wherein said protein has a glycerol-3-phosphate dehydrogenase activity;

(k) a polynucleotide comprising a polynucleotide encoding a protein having 90% or higher identity with the amino acid sequence of SEQ ID NO: 10, and having a glycerol-3-phosphate dehydrogenase activity; and

(l) a polynucleotide comprising a polynucleotide which hybridizes to a polynucleotide consisting of a nucleotide sequence of SEQ ID NO: 9 or which hybridizes to a polynucleotide consisting of a nucleotide sequence complementary to the nucleotide sequence of SEQ ID NO: 9, under high stringent conditions, which encodes a protein having a glycerol-3-phosphate dehydrogenase activity.

According to the method for producing alcoholic beverages using transformed yeast of the present invention, alcoholic beverages with superior body and mellowness can be produced because the method can control the amount of glycerol, which provides body and mellowness to the product. Further, the transformed yeast of the present invention is rich in glycerol content. A yeast with elevated glycerol production is thought to be able to respond promptly to stresses including osmotic stress, and thus has a superior low-temperature storage property, frozen storage property or drying-resistant property. Moreover, fermentation time can be reduced in high concentration brewing because transformed yeast of the present invention has a osmotic-pressure resistant property.

### BRIEF DESCRIPTION OF DRAWINGS

FIG. 1 shows the cell growth with time upon beer fermentation test. The horizontal axis represents fermentation time while the vertical axis represents optical density at 660 nm (OD660).

FIG. 2 shows the extract (sugar) consumption with time upon beer fermentation test. The horizontal axis represents fermentation time while the vertical axis represents apparent extract concentration (w/w %).

FIG. 3 shows the expression profile of non-ScGPD1 gene in yeasts upon beer fermentation test. The horizontal axis represents fermentation time while the vertical axis represents the intensity of detected signal.

FIG. 4 shows the cell growth with time upon beer fermentation test. The horizontal axis represents fermentation time while the vertical axis represents optical density at 660 nm (OD660).

FIG. 5 shows the extract (sugar) consumption with time upon beer fermentation test. The horizontal axis represents fermentation time while the vertical axis represents apparent extract concentration (w/w %).

FIG. 6 shows the expression profile of non-ScGPD2 gene in yeasts upon beer fermentation test. The horizontal axis represents fermentation time while the vertical axis represents the intensity of detected signal.

FIG. 7 shows the cell growth with time upon beer fermentation test. The horizontal axis represents fermentation time while the vertical axis represents optical density at 660 nm (OD660).

FIG. 8 shows the extract (sugar) consumption with time upon beer fermentation test. The horizontal axis represents fermentation time while the vertical axis represents apparent extract concentration (w/w %).

FIG. 9 shows production amount of glycerol with time upon beer fermentation test. The horizontal axis represents fermentation time while the vertical axis represents glycerol level (g/L).

FIG. 10 shows the cell growth with time upon beer fermentation test. The horizontal axis represents fermentation time while the vertical axis represents optical density at 660 nm (OD660).

FIG. 11 shows the extract (sugar) consumption with time upon beer fermentation test. The horizontal axis represents fermentation time while the vertical axis represents apparent extract concentration (w/w %).

FIG. 12 shows production amount of ethanol with time upon beer fermentation test. The horizontal axis represents fermentation time while the vertical axis represents ethanol level (g/L).

### BEST MODES FOR CARRYING OUT THE INVENTION

The present inventors conceived that it is possible to produce glycerol more effectively by increasing an activity of glycerol-3-phosphate dehydrogenase of yeast. The present inventors have studied based on this conception and as a result, isolated and identified non-ScGPD1 gene and non-ScGPD2 gene encoding glycerol-3-phosphate dehydrogenases of lager brewing yeast based on the lager brewing yeast genome information mapped according to the method disclosed in Japanese Patent Application Laid-Open No. 2004-283169. The nucleotide sequences of the genes are represented by SEQ ID NO: 1 and SEQ ID NO: 3. Further, amino acid sequences of proteins encoded by the genes are represented by SEQ ID NO: 2 and SEQ ID NO: 4, respectively. Moreover, the present inventors isolated and identified ScGPD1 gene encoding glycerol-3-phosphate dehydrogenase of lager brewing yeast. The nucleotide sequence of the gene is represented by SEQ ID NO: 9. Further, amino acid sequence of a protein encoded by the gene is represented by SEQ ID NO: 10.

1. Polynucleotide of the invention

First of all, the present invention provides (a) a polynucleotide comprising a polynucleotide of the nucleotide sequence of SEQ ID NO: 1, SEQ ID NO: 3 or SEQ ID NO: 9; and (b) a polynucleotide comprising a polynucleotide encoding a protein of the amino acid sequence of SEQ ID NO: 2, SEQ ID NO: 4 or SEQ ID NO: 10. The polynucleotide can be DNA or RNA.

The target polynucleotide of the present invention is not limited to the polynucleotide encoding a glycerol-3-phosphate dehydrogenase described above and may include other polynucleotides encoding proteins having equivalent functions to said protein. Proteins with equivalent functions include, for example, (c) a protein of an amino acid sequence of SEQ ID NO: 2, SEQ ID NO: 4 or SEQ ID NO: 10 with one or more amino acids thereof being deleted, substituted, inserted and/or added and having a glycerol-3-phosphate dehydrogenase activity.

Such proteins include a protein consisting of an amino acid sequence of SEQ ID NO: 2, SEQ ID NO: 4 or SEQ ID NO: 10 with, for example, 1 to 100, 1 to 90, 1 to 80, 1 to 70, 1 to 60, 1 to 50, 1 to 40, 1 to 39, 1 to 38, 1 to 37, 1 to 36, 1 to 35, 1 to 34, 1 to 33, 1 to 32, 1 to 31, 1 to 30, 1 to 29, 1 to 28, 1 to 27, 1 to 26, 1 to 25, 1 to 24, 1 to 23, 1 to 22, 1 to 21, 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6 (1 to several amino acids), 1 to 5, 1 to 4, 1 to 3, 1 to 2, or 1 amino acid residues thereof being deleted, substituted, inserted and/or added and having a glycerol-3-phosphate dehydrogenase activity. In general, the number of deletions, substitutions, insertions, and/or additions is preferably smaller. In addition, such proteins include (d) a protein having an amino acid sequence with about 60% or higher, about 70% or higher, 71% or higher, 72% or higher, 73% or higher, 74% or higher, 75% or higher, 76% or higher, 77% or higher, 78% or higher, 79% or higher, 80% or higher, 81% or higher, 82% or higher, 83% or higher, 84% or higher, 85% or higher, 86% or higher, 87% or higher, 88% or higher, 89% or higher, 90% or higher, 91% or higher, 92% or higher, 93% or higher, 94% or higher, 95% or higher, 96% or higher, 97% or higher, 98% or higher, 99% or higher, 99.1% or higher, 99.2% or higher, 99.3% or higher, 99.4% or higher, 99.5% or higher, 99.6% or higher, 99.7% or higher, 99.8% or higher, or 99.9% or higher identity with the amino acid sequence of SEQ ID NO: 2, SEQ ID NO: 4 or SEQ ID NO: 10, and having a glycerol-3-phosphate dehydrogenase activity. In general, the percentage identity is preferably higher.

Glycerol-3-phosphate dehydrogenase activity may be measured, for example, by a method described in Yeast 12: 1331-1337, 1996.

Furthermore, the present invention also contemplates (e) a polynucleotide comprising a polynucleotide which hybridizes to a polynucleotide consisting of a nucleotide sequence complementary to the nucleotide sequence of SEQ ID NO: 1, SEQ ID NO: 3 or SEQ ID NO: 9 under stringent conditions and which encodes a glycerol-3-phosphate dehydrogenase; and (f) a polynucleotide comprising a polynucleotide which hybridizes to a polynucleotide complementary to a nucleotide sequence encoding a protein consisting of an amino acid sequence of SEQ ID NO: 2, SEQ ID NO: 4 or SEQ ID NO: 10 under stringent conditions, and which encodes a glycerol-3-phosphate dehydrogenase.

Herein, “a polynucleotide that hybridizes under stringent conditions” refers to nucleotide sequence, such as a DNA, obtained by a colony hybridization technique, a plaque hybridization technique, a southern hybridization technique or the like using all or part of polynucleotide of a nucleotide sequence complementary to the nucleotide sequence of SEQ ID NO: 1, SEQ ID NO: 3 or SEQ ID NO: 9 or polynucleotide encoding the amino acid sequence of SEQ ID NO: 2, SEQ ID NO: 4 or SEQ ID NO: 10 as a probe. The hybridization method may be a method described, for example, in MOLECULAR CLONING 3rd Ed., CURRENT PROTOCOLS IN MOLECULAR BIOLOGY, John Wiley & Sons 1987-1997, and so on.

The term “stringent conditions” as used herein may be any of low stringency conditions, moderate stringency conditions or high stringency conditions. “Low stringency conditions” are, for example, 5×SSC, 5×Denhardt's solution, 0.5% SDS, 50% formamide at 32° C. “Moderate stringency conditions” are, for example, 5×SSC, 5×Denhardt's solution, 0.5% SDS, 50% formamide at 42° C. “High stringency conditions” are, for example, 5×SSC, 5×Denhardt's solution, 0.5% SDS, 50% formamide at 50° C. Under these conditions, a polynucleotide, such as a DNA, with higher homology is expected to be obtained efficiently at higher temperature, although multiple factors are involved in hybridization stringency including temperature, probe concentration, probe length, ionic strength, time, salt concentration and others, and one skilled in the art may appropriately select these factors to realize similar stringency.

When a commercially available kit is used for hybridization, for example, Alkphos Direct Labeling Reagents (Amersham Pharmacia) may be used. In this case, according to the attached protocol, after incubation with a labeled probe overnight, the membrane is washed with a primary wash buffer containing 0.1% (w/v) SDS at 55° C., thereby detecting hybridized polynucleotide, such as DNA.

Other polynucleotides that can be hybridized include polynucleotides having about 60% or higher, about 70% or higher, 71% or higher, 72% or higher, 73% or higher, 74% or higher, 75% or higher, 76% or higher, 77% or higher, 78% or higher, 79% or higher, 80% or higher, 81% or higher, 82% or higher, 83% or higher, 84% or higher, 85% or higher, 86% or higher, 87% or higher, 88% or higher, 89% or higher, 90% or higher, 91% or higher, 92% or higher, 93% or higher, 94% or higher, 95% or higher, 96% or higher, 97% or higher, 98% or higher, 99% or higher, 99.1% or higher, 99.2% or higher, 99.3% or higher, 99.4% or higher, 99.5% or higher, 99.6% or higher, 99.7% or higher, 99.8% or higher or 99.9% or higher identity to polynucleotide encoding the amino acid sequence of SEQ ID NO: 2, SEQ ID NO: 4 or SEQ ID NO: 10 as calculated by homology search software, such as FASTA and BLAST using default parameters.

Identity between amino acid sequences or nucleotide sequences may be determined using algorithm BLAST by Karlin and Altschul (Proc. Natl. Acad. Sci. USA, 87: 2264-2268, 1990; Proc. Natl. Acad. Sci. USA, 90: 5873, 1993). Programs called BLASTN and BLASTX based on BLAST algorithm have been developed (Altschul S F et al., J. Mol. Biol. 215: 403, 1990). When a nucleotide sequence is sequenced using BLASTN, the parameters are, for example, score=100 and word length=12. When an amino acid sequence is sequenced using BLASTX the parameters are, for example, score=50 and word length=3. When BLAST and Gapped BLAST programs are used, default parameters for each of the programs are employed.

### 2. Protein of the Present Invention

The present invention also provides proteins encoded by any of the polynucleotides (a) to (1) above. A preferred protein of the present invention comprises an amino acid sequence of SEQ ID NO: 2, SEQ ID NO: 4 or SEQ ID NO: 10 with one or several amino acids thereof being deleted, substituted, inserted and/or added, and having a glycerol-3-phosphate dehydrogenase activity.

Such protein includes those having an amino acid sequence of SEQ ID NO: 2, SEQ ID NO: 4 or SEQ ID NO: 10 with amino acid residues thereof of the number mentioned above being deleted, substituted, inserted and/or added and having a glycerol-3-phosphate dehydrogenase activity. In addition, such protein includes those having homology as described above with the amino acid sequence of SEQ ID NO: 2, SEQ ID NO: 4 or SEQ ID NO: 10 and having a glycerol-3-phosphate dehydrogenase activity.

Such proteins may be obtained by employing site-directed mutation described, for example, in MOLECULAR CLONING 3rd Ed., CURRENT PROTOCOLS IN MOLECULAR BIOLOGY, Nuc. Acids. Res., 10: 6487 (1982), Proc. Natl. Acad. Sci. USA 79: 6409 (1982), Gene 34: 315 (1985), Nuc. Acids. Res., 13: 4431 (1985), Proc. Natl. Acad. Sci. USA 82: 488 (1985).

Deletion, substitution, insertion and/or addition of one or more amino acid residues in an amino acid sequence of the protein of the invention means that one or more amino acid residues are deleted, substituted, inserted and/or added at any one or more positions in the same amino acid sequence. Two or more types of deletion, substitution, insertion and/or addition may occur concurrently.

Hereinafter, examples of mutually substitutable amino acid residues are enumerated. Amino acid residues in the same group are mutually substitutable. The groups are provided below.

Group A: leucine, isoleucine, norleucine, valine, norvaline, alanine, 2-aminobutanoic acid, methionine, o-methylserine, t-butylglycine, t-butylalanine, cyclohexylalanine; Group B: asparatic acid, glutamic acid, isoasparatic acid, isoglutamic acid, 2-aminoadipic acid, 2-aminosuberic acid; Group C: asparagine, glutamine; Group D: lysine, arginine, ornithine, 2,4-diaminobutanoic acid, 2,3-diaminopropionic acid; Group E: proline, 3-hydroxyproline, 4-hydroxyproline; Group F: serine, threonine, homoserine; and Group G: phenylalanine, tyrosine.

The protein of the present invention may also be produced by chemical synthesis methods such as Fmoc method (fluorenylmethyloxycarbonyl method) and tBoc method (t-butyloxycarbonyl method). In addition, peptide synthesizers available from for example, Advanced ChemTech, PerkinElmer, Pharmacia, Protein Technology Instrument, Synthecell-Vega, PerSeptive, Shimazu Corp. can also be used for chemical synthesis.

3. Vector of the Invention and Yeast Transformed with the Vector

The present invention then provides a vector comprising the polynucleotide described above. The vector of the present invention is directed to a vector including any of the polynucleotides described in (a) to (1) above. Generally, the vector of the present invention comprises an expression cassette including as components (x) a promoter that can transcribe in a yeast cell; (y) a polynucleotide described in any of (a) to (1) above that is linked to the promoter in sense or antisense direction; and (z) a signal that functions in the yeast with respect to transcription termination and polyadenylation of RNA molecule. Further, in order to highly express the protein of the invention, these polynucleotides are preferably introduced in the sense direction to the promoter to promote expression of the polynucleotide (DNA) described in any of (a) to (1) above.

A vector introduced in the yeast may be any of a multicopy type (YEp type), a single copy type (YCp type), or a chromosome integration type (YIp type). For example, YEp24 (J. R. Broach et al., EXPERMENTAL MANIPULATION OF GENE EXPRESSION, Academic Press, New York 83, 1983) is known as a YEp type vector, YCp50 (M. D. Rose et al., Gene 60: 237, 1987) is known as a YCp type vector, and YIp5 (K. Struhl et al., Proc. Natl. Acad. Sci. USA, 76: 1035, 1979) is known as a YIp type vector, all of which are readily available.

Promoters/terminators for adjusting gene expression in yeast may be in any combination as long as they function in the brewery yeast and they are not influenced by constituents in fermentation broth. For example, a promoter of glyceraldehydes 3-phosphate dehydrogenase gene (TDH3), or a promoter of 3-phosphoglycerate kinase gene (PGK1) may be used. These genes have previously been cloned, described in detail, for example, in M. F. Tuite et al., EMBO J., 1, 603 (1982), and are readily available by known methods.

Since an auxotrophy marker cannot be used as a selective marker upon transformation for a brewery yeast, for example, a geneticin-resistant gene (G418r), a copper-resistant gene (CUP1) (Marin et al., Proc. Natl. Acad. Sci. USA, 81, 337 1984) or a cerulenin-resistant gene (fas2m, PDR4) (Junji Inokoshi et al., Biochemistry, 64, 660, 1992; and Hussain et al., Gene, 101: 149, 1991, respectively) may be used.

A vector constructed as described above is introduced into a host yeast. Examples of the host yeast include any yeast that can be used for brewing, for example, brewery yeasts for beer, wine and sake and so on. Specifically, yeasts such as genus Saccharomyces may be used. According to the present invention, a lager brewing yeast, for example, Saccharomyces pastorianus W34/70, etc., Saccharomyces carlsbergensis NCYC453 or NCYC456, etc., or Saccharomyces cerevisiae NBRC1951, NBRC1952, NBRC1953 or NBRC1954, etc., may be used. In addition, whisky yeasts such as Saccharomyces cerevisiae NCYC90, wine yeasts such as wine yeasts #1, 3 and 4 from the Brewing Society of Japan, and sake yeasts such as sake yeast #7 and 9 from the Brewing Society of Japan may also be used but not limited thereto. In the present invention, lager brewing yeasts such as Saccharomyces pastorianus may be used preferably.

A yeast transformation method may be a generally used known method. For example, methods that can be used include but not limited to an electroporation method (Meth. Enzym., 194: 182 (1990)), a spheroplast method (Proc. Natl. Acad. Sci. USA, 75: 1929 (1978)), a lithium acetate method (J. Bacteriology, 153: 163 (1983)), and methods described in Proc. Natl. Acad. Sci. USA, 75: 1929 (1978), METHODS IN YEAST GENETICS, 2000 Edition: A Cold Spring Harbor Laboratory Course Manual.

More specifically, a host yeast is cultured in a standard yeast nutrition medium (e.g., YEPD medium (Genetic Engineering. Vol. 1, Plenum Press, New York 117 (1979)), etc.) such that OD600 nm will be 1 to 6. This culture yeast is collected by centrifugation, washed and pre-treated with alkali metal ion, preferably lithium ion at a concentration of about 1 to 2 M. After the cell is left to stand at about 30° C. for about 60 minutes, it is left to stand with DNA to be introduced (about 1 to 20 μg) at about 30° C. for about another 60 minutes. Polyethyleneglycol, preferably about 4,000 Dalton of polyethyleneglycol, is added to a final concentration of about 20% to 50%. After leaving at about 30° C. for about 30 minutes, the cell is heated at about 42° C. for about 5 minutes. Preferably, this cell suspension is washed with a standard yeast nutrition medium, added to a predetermined amount of fresh standard yeast nutrition medium and left to stand at about 30° C. for about 60 minutes. Thereafter, it is seeded to a standard agar medium containing an antibiotic or the like as a selective marker to obtain a transformant.

Other general cloning techniques may be found, for example, in MOLECULAR CLONING 3rd Ed., and METHODS IN YEAST GENETICS, A LABORATORY MANUAL (Cold Spring Harbor Laboratory Press, Cold Spring Harbor, N.Y.).

### 4. Method of Producing Alcoholic Beverages According to the Present Invention and Alcoholic Beverages Produced by the Method

The vector of the present invention described above is introduced into a yeast suitable for brewing a target alcoholic product. This yeast can be used to produce a desired alcoholic beverage with an elevated content of glycerol. In addition, yeasts to be selected by the yeast assessment method of the present invention described below can also be used. Moreover, fermentation time can be reduced in high concentration brewing because the yeast has a osmotic pressure resistant property.

The target alcoholic beverages include, for example, but not limited to beer, beer-taste beverages such as sparkling liquor (happoushu), wine, whisky, sake and the like. Further, according to the present invention, desired alcoholic beverages with reduced glycerol level can be produced using brewery yeast in which the expression of the target gene was suppressed, if needed. That is to say, desired kind of alcoholic beverages with controlled (elevated or reduced) level of glycerol can be produced by controlling (elevating or reducing) production amount of glycerol using yeasts into which the vector of the present invention was introduced described above, yeasts in which expression of the polynucleotide (DNA) of the present invention described above was suppressed or yeasts selected by the yeast assessment method of the invention described below for fermentation to produce alcoholic beverages.

In order to produce these alcoholic beverages, a known technique can be used except that a brewery yeast obtained according to the present invention is used in the place of a parent strain. Since materials, manufacturing equipment, manufacturing control and the like may be exactly the same as the conventional ones, there is no need of increasing the cost for producing alcoholic beverages with an elevated content of glycerol. Thus, according to the present invention, alcoholic beverages with excellent body and mellowness can be produced using existing facility without increasing the cost. Moreover, cost reduction is expected because fermentation time can be reduced in high concentration brewing using existing facility.

### 5. Yeast Assessment Method of the Invention

The present invention relates to a method for assessing a test yeast for its glycerol producing ability by using a primer or a probe designed based on a nucleotide sequence of a glycerol-3-phosphate dehydrogenase gene having the nucleotide sequence of SEQ ID NO: 1, SEQ ID NO: 3 or SEQ ID NO: 9. General technique for such assessment method is known and is described in, for example, WO01/040514, Japanese Laid-Open Patent Application No. H8-205900 or the like. This assessment method is described in below.

First, genome of a test yeast is prepared. For this preparation, any known method such as Hereford method or potassium acetate method may be used (e.g., METHODS IN YEAST GENETICS, Cold Spring Harbor Laboratory Press, 130 (1990)). Using a primer or a probe designed based on a nucleotide sequence (preferably, ORF sequence) of the glycerol-3-phosphate dehydrogenase gene, the existence of the gene or a sequence specific to the gene is determined in the test yeast genome obtained. The primer or the probe may be designed according to a known technique.

Detection of the gene or the specific sequence may be carried out by employing a known technique. For example, a polynucleotide including part or all of the specific sequence or a polynucleotide including a nucleotide sequence complementary to said nucleotide sequence is used as one primer, while a polynucleotide including part or all of the sequence upstream or downstream from this sequence or a polynucleotide including a nucleotide sequence complementary to said nucleotide sequence, is used as another primer to amplify a nucleic acid of the yeast by a PCR method, thereby determining the existence of amplified products and molecular weight of the amplified products. The number of bases of polynucleotide used for a primer is generally 10 base pairs (bp) or more, and preferably 15 to 25 bp. In general, the number of bases between the primers is suitably 300 to 2000 bp.

The reaction conditions for PCR are not particularly limited but may be, for example, a denaturation temperature of 90 to 95° C., an annealing temperature of 40 to 60° C., an elongation temperature of 60 to 75° C., and the number of cycle of 10 or more. The resulting reaction product may be separated, for example, by electrophoresis using agarose gel to determine the molecular weight of the amplified product. This method allows prediction and assessment of the drying-resistant property and/or low-temperature storage-resistant property of yeast as determined by whether the molecular weight of the amplified product is a size that contains the DNA molecule of the specific part. In addition, by analyzing the nucleotide sequence of the amplified product, the property may be predicted and/or assessed more precisely.

Moreover, in the present invention, a test yeast is cultured to measure an expression level of the glycerol-3-phosphate dehydrogenase gene having the nucleotide sequence of SEQ ID NO: 1, SEQ ID NO: 3 or SEQ ID NO: 9 to assess the test yeast for its glycerol producing ability. Measurement of expression level of the glycerol-3-phosphate dehydrogenase gene can be performed by culturing test yeast and then quantifying mRNA or a protein resulting from the gene. The quantification of mRNA or protein may be carried out by employing a known technique. For example, mRNA may be quantified, by Northern hybridization or quantitative RT-PCR, while protein may be quantified, for example, by Western blotting (CURRENT PROTOCOLS IN MOLECULAR BIOLOGY, John Wiley & Sons 1994-2003).

Furthermore, test yeasts are cultured and expression levels of the glycerol-3-phosphate dehydrogenase gene having the nucleotide sequence of SEQ ID NO: 1, SEQ ID NO: 3 or SEQ ID NO: 9 are measured to select a test yeast with the gene expression level according to the target glycerol producing ability, thereby a yeast favorable for brewing desired alcoholic beverages can be selected. In addition, a reference yeast and a test yeast may be cultured so as to measure and compare the expression level of the gene in each of the yeasts, thereby a favorable test yeast can be selected. More specifically, for example, a reference yeast and one or more test yeasts are cultured and an expression level of the glycerol-3-phosphate dehydrogenase gene having the nucleotide sequence of SEQ ID NO: 1, SEQ ID NO: 3 or SEQ ID NO: 9 is measured in each yeast. By selecting a test yeast with the gene expressed higher than that in the reference yeast, a yeast suitable for brewing desired alcoholic beverages can be selected.

Alternatively, test yeasts are cultured and a yeast with a high or low glycerol producing ability, or a high or low glycerol-3-phosphate dehydrogenase activity is selected, thereby a yeast suitable for brewing desired alcoholic beverages can be selected.

In these cases, the test yeasts or the reference yeast may be, for example, a yeast introduced with the vector of the invention, an artificially mutated yeast or a naturally mutated yeast. The glycerol producing ability can be measured by, for example, by a method described in Methods of Enzymatic Analysis, vol. 4 1825-1831, 1974. The glycerol-3-phosphate dehydrogenase activity can be measured by, for example, a method described in Yeast 12: 1331-1337, 1996). The mutation treatment may employ any methods including, for example, physical methods such as ultraviolet irradiation and radiation irradiation, and chemical methods associated with treatments with drugs such as EMS (ethylmethane sulphonate) and N-methyl-N-nitrosoguanidine (see, e.g., Yasuji Oshima Ed., BIOCHEMISTRY EXPERIMENTS vol. 39, Yeast Molecular Genetic Experiments, pp. 67-75, JSSP).

In addition, examples of yeasts used as the reference yeast or the test yeasts include any yeast, for example, brewery yeasts for beer, wine, sake and the like. More specifically, yeasts such as genus Saccharomyces may be used (e.g., S. pastorianus, S. cerevisiae, and S. carlsbergensis). According to the present invention, a lager brewing yeast, for example, Saccharomyces pastorianus W34/70; Saccharomyces carlsbergensis NCYC453 or NCYC456; or Saccharomyces cerevisiae NBRC1951, NBRC1952, NBRC1953 or NBRC1954, etc., may be used. Further, wine yeasts such as wine yeasts #1, 3 and 4 from the Brewing Society of Japan; and sake yeasts such as sake yeast #7 and 9 from the Brewing Society of Japan may also be used but not limited thereto. In the present invention, lager brewing yeasts such as Saccharomyces pastorianus may preferably be used. The reference yeast and the test yeasts may be selected from the above yeasts in any combination.

### EXAMPLES

Hereinafter, the present invention will be described in more detail with reference to working examples. The present invention, however, is not limited to the examples described below.

### Example 1

### Cloning of Glycerol-3-phosphate dehydrogenase Gene (non-ScGPD1)

A glycerol-3-phosphate dehydrogenase gene of lager brewing yeast (non-ScGPD1) (SEQ ID NO: 1) was found as a result of a search utilizing the comparison database described in Japanese Patent Application Laid-Open No. 2004-283169. Based on the acquired nucleotide sequence information, primers non-ScGPD1_for (SEQ ID NO: 5) and non-ScGPD1_rv (SEQ ID NO: 6) were designed to amplify the full-length of the gene. PCR was carried out using chromosomal DNA of a genome sequencing strain, Saccharomyces pastorianus Weihenstephan 34/70 (sometimes abbreviated as “W34/70 strain”), as a template to obtain DNA fragments (about 1.2 kb) including the full-length gene of non-ScGPD1.

The non-ScGPD1 gene fragments thus obtained were inserted into pCR2.1-TOPO vector (Invitrogen) by TA cloning. The nucleotide sequences of the non-ScGPD1 gene were analyzed by Sanger's method (F. Sanger, Science, 214: 1215, 1981) to confirm the nucleotide sequence.

### Example 2

### Analysis of Expression of Non-ScGPD1 Gene During Beer Fermentation

A beer fermentation test was conducted using a lager brewing yeast, Saccharomyces pastorianus W34/70, and mRNA extracted from the lager brewing yeast during fermentation was detected by a beer yeast DNA microarray.

| | |
|---|---|
| Wort extract concentration | 12.69% |
| Wort content | 70 L |
| Wort dissolved oxygen concentration | 8.6 ppm |
| Fermentation temperature | 15° C. |
| Yeast pitching rate | 12.8 × 10⁶ cells/mL |

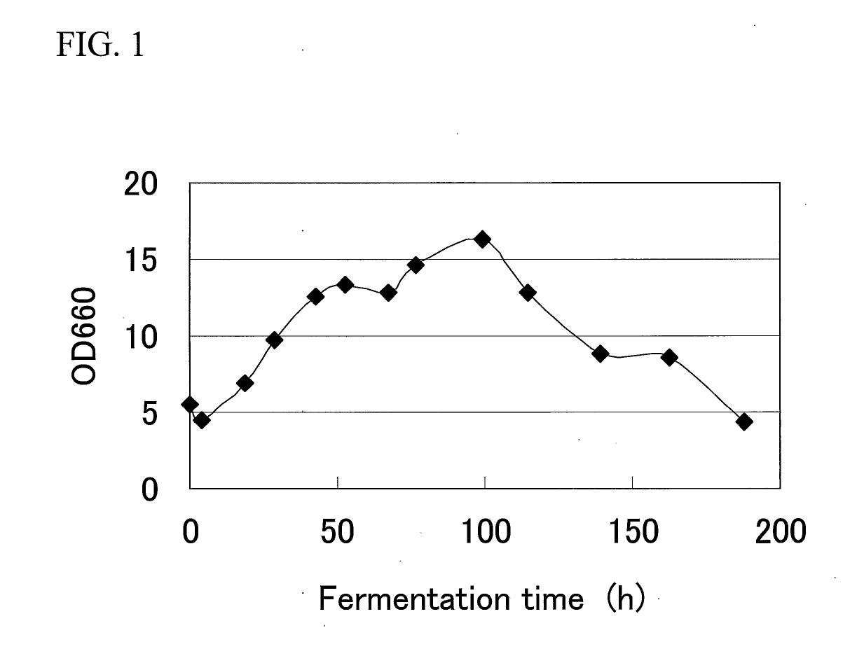

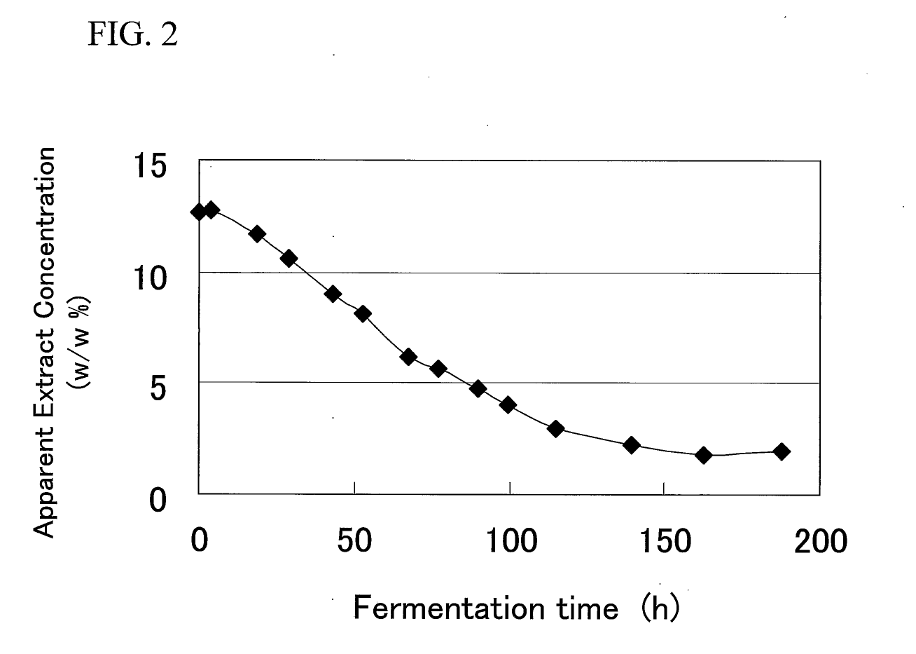

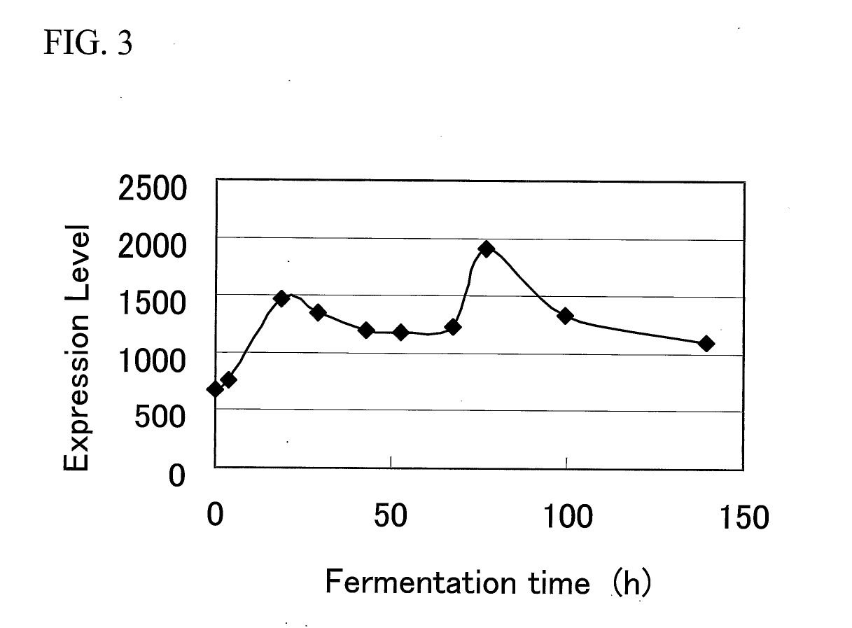

The fermentation liquor was sampled over time, and the time-course changes in amount of yeast cell growth (FIG. 1) and apparent extract concentration (FIG. 2) were observed. Simultaneously, yeast cells were sampled to prepare mRNA, and the prepared mRNA was labeled with biotin and was hybridized to a beer yeast DNA microarray. The signal was detected using GeneChip Operating system (GCOS; GeneChip Operating Software 1.0, manufactured by Affymetrix Co). Expression pattern of the non-ScGPD1 gene is shown in FIG. 3. This result confirmed the expression of the non-ScGPD1 gene in the general beer fermentation.

### Example 3

### Construction of Non-ScGPD1 Highly Expressed Strain

The non-ScGPD1/pCR2.1-TOPO obtained by the method described in Example 1 was digested with the restriction enzymes SacI and BamHI to prepare a DNA fragment containing the entire length of the protein-encoding region. This fragment was ligated to pYEGNot treated with the restriction enzymes SacI and BamHI, thereby constructing the non-ScGPD1 high expression vector non-ScGPD1/pYEGNot. pYEGNot is a YEp-type yeast expression vector. A gene inserted is highly expressed by the pyruvate kinase gene PYK1 promoter. The geneticin-resistant gene G418^{r} is included as the selectable marker in the yeast, and the ampicillin-resistant gene Amp^{r} as the selectable marker in Escherichia coli.

Using the high expression vector prepared by the above method, the Saccharomyces pastorianus Weihenstephan 34/70 strain, a Saccharomyces cerevisiae BH174 strain or a Saccharomyces cerevisiae BH172 strain was transformed by the method described in Japanese Patent Application Laid-open No. H07-303475. The transformants were selected on a YPD plate medium (1% yeast extract, 2% polypeptone, 2% glucose and 2% agar) containing 300 mg/L of geneticin.

### Example 4

### Cloning of Glycerol-3-Phosphate Dehydrogenase Gene (Non-ScGPD2)

A glycerol-3-phosphate dehydrogenase gene of lager brewing yeast (non-ScGPD2) (SEQ ID NO: 3) was found as a result of a search utilizing the comparison database described in Japanese Patent Application Laid-Open No. 2004-283169. Based on the acquired nucleotide sequence information, primers non-ScGPD2_for (SEQ ID NO: 7) and non-ScGPD2_rv (SEQ ID NO: 8) were designed to amplify the full-length of the gene. PCR was carried out using chromosomal DNA of a genome sequencing strain, Saccharomyces pastorianus Weihenstephan 34/70, as a template to obtain DNA fragments (about 1.3 kb) including the full-length gene of non-ScGPD2.

The non-ScGPD2 gene fragments thus obtained were inserted into pCR2.1-TOPO vector (Invitrogen) by TA cloning. The nucleotide sequences of the non-ScGPD2 gene were analyzed by Sanger's method (F. Sanger, Science, 214: 1215, 1981) to confirm the nucleotide sequence.

### Example 5

### Analysis of Expression of Non-ScGPD2 Gene During Beer Fermentation

A beer fermentation test was conducted using a lager brewing yeast, Saccharomyces pastorianus W34/70, and mRNA extracted from the lager brewing yeast during fermentation was detected by a beer yeast DNA microarray.

| | |
|---|---|
| Wort extract concentration | 12.69% |
| Wort content | 70 L |
| Wort dissolved oxygen concentration | 8.6 ppm |
| Fermentation temperature | 15° C. |
| Yeast pitching rate | 12.8 × 10⁶ cells/mL |

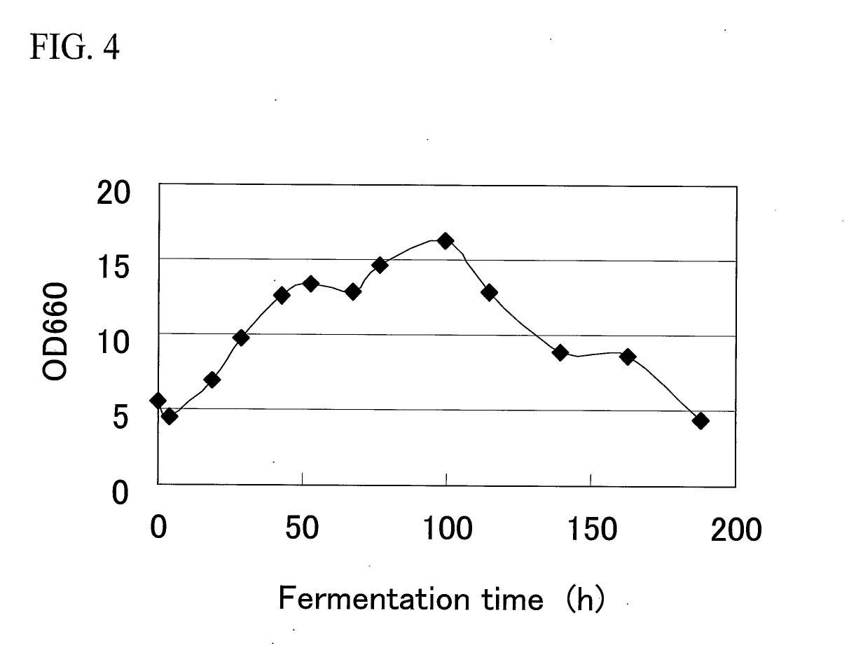

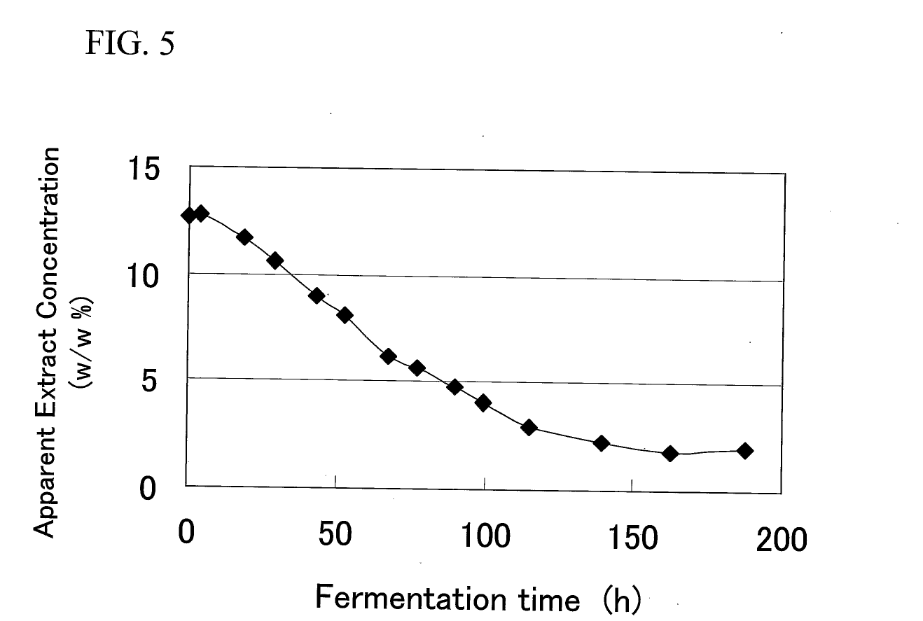

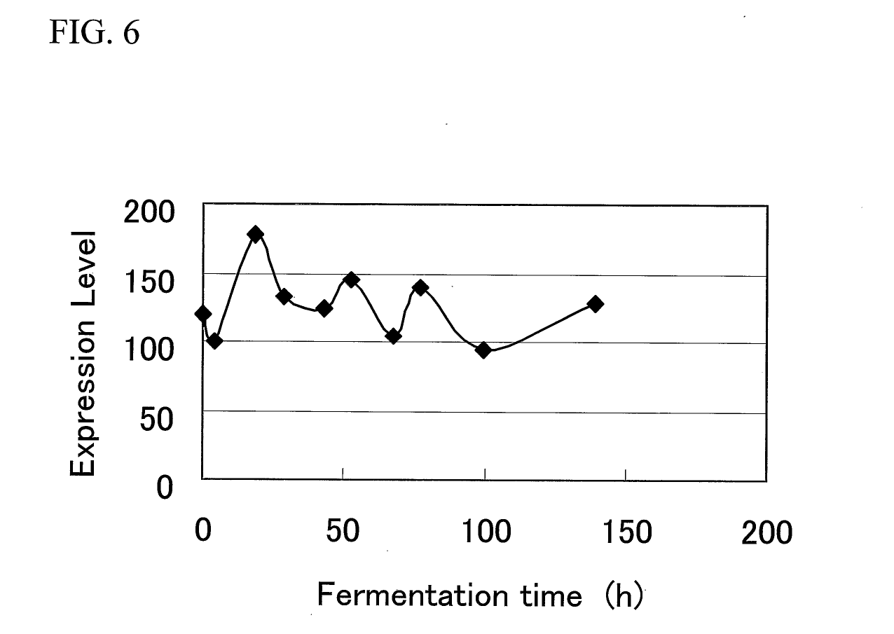

The fermentation liquor was sampled over time, and the time-course changes in amount of yeast cell growth (FIG. 4) and apparent extract concentration (FIG. 5) were observed. Simultaneously, yeast cells were sampled to prepare mRNA, and the prepared mRNA was labeled with biotin and was hybridized to a beer yeast DNA microarray. The signal was detected using GeneChip Operating system (GCOS; GeneChip Operating Software 1.0, manufactured by Affymetrix Co). Expression pattern of the non-ScGPD2 gene is shown in FIG. 6. This result confirmed the expression of the non-ScGPD2 gene in the general beer fermentation.

### Example 6

### Analysis of Amount of Glycerol Production During Beer Fermentation

A fermentation test was carried out under the following conditions using the parent strain (34/70 strain) and the non-ScGPD1 highly expressed strain obtained by the method described in Example 3.

| | |
|---|---|
| Wort extract concentration | 12% |
| Wort content | 1 L |
| Wort dissolved oxygen concentration | approx. 10 ppm |
| Fermentation temperature | 15° C. (fixed) |
| Yeast pitching rate | 5 g wet yeast cells/L of wort |

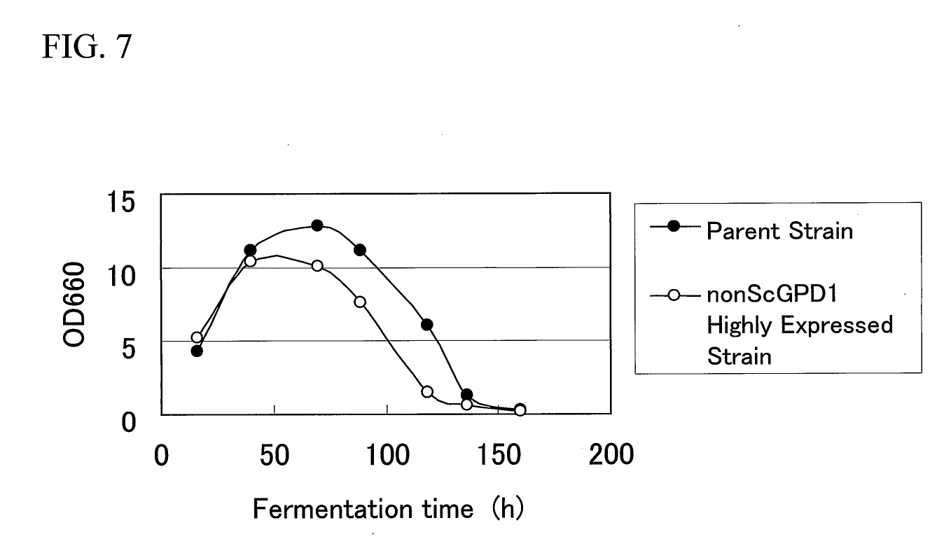

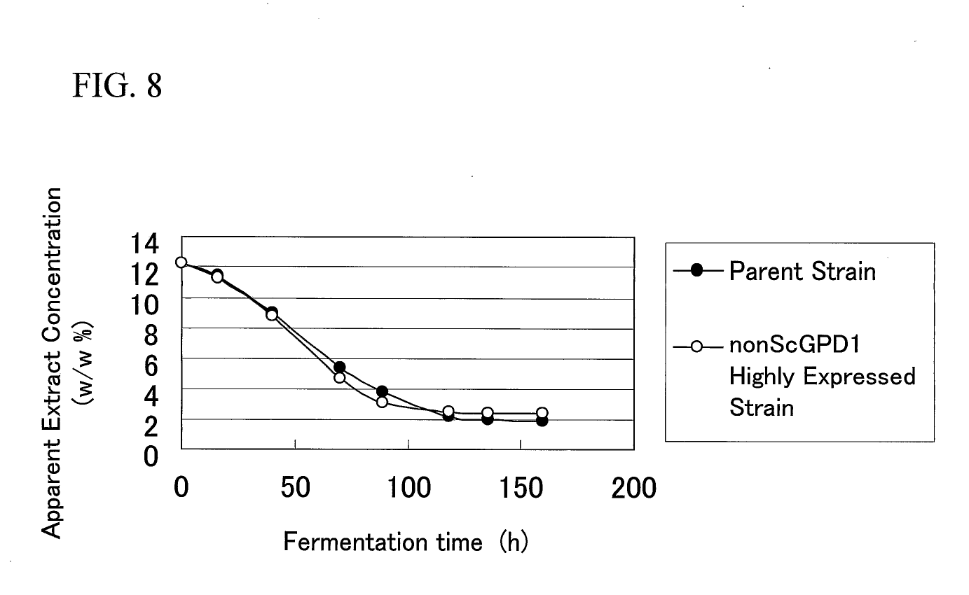

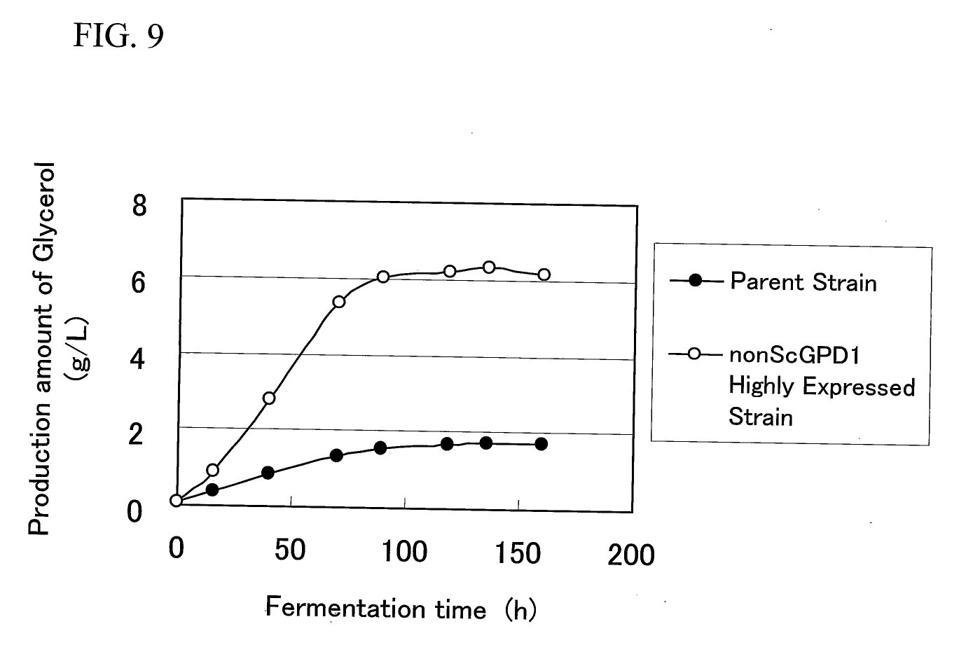

The fermentation broth was sampled over time, and the change over time in the yeast growth rate (OD660) (FIG. 7), the amount of extract consumption (FIG. 8) and the amount of glycerol production (FIG. 9) were determined. Glycerol in the fermentation broth was quantified using F-kit glycerol (product number 148270, manufactured by Roche) (see Method of Enzymatic Analysis, vol. 4 1825-1831, 1974, etc.). The amount of glycerol in the fermentation broth at the completion of fermentation was 1.7 g/L for the parent strain, and was 6.2 g/L for non-ScGPD1 highly expressed strain, which was about 3.6-fold of the parent strain.

### Example 7

### Beer Fermentation Test Using High Concentration Wort

A fermentation test was carried out under the following conditions using the parent strain (34/70 strain) and the non-ScGPD1 highly expressed strain obtained by the method described in Example 3.

| | |
|---|---|
| Wort extract concentration | 19.3% (saccharified syrup |
| | was added to 12% wort) |
| Wort content | 1 L |
| Wort dissolved oxygen concentration | approx. 10 ppm |
| Fermentation temperature | 15° C. (fixed) |
| Yeast pitching rate | 5 g wet yeast cells/L of wort |

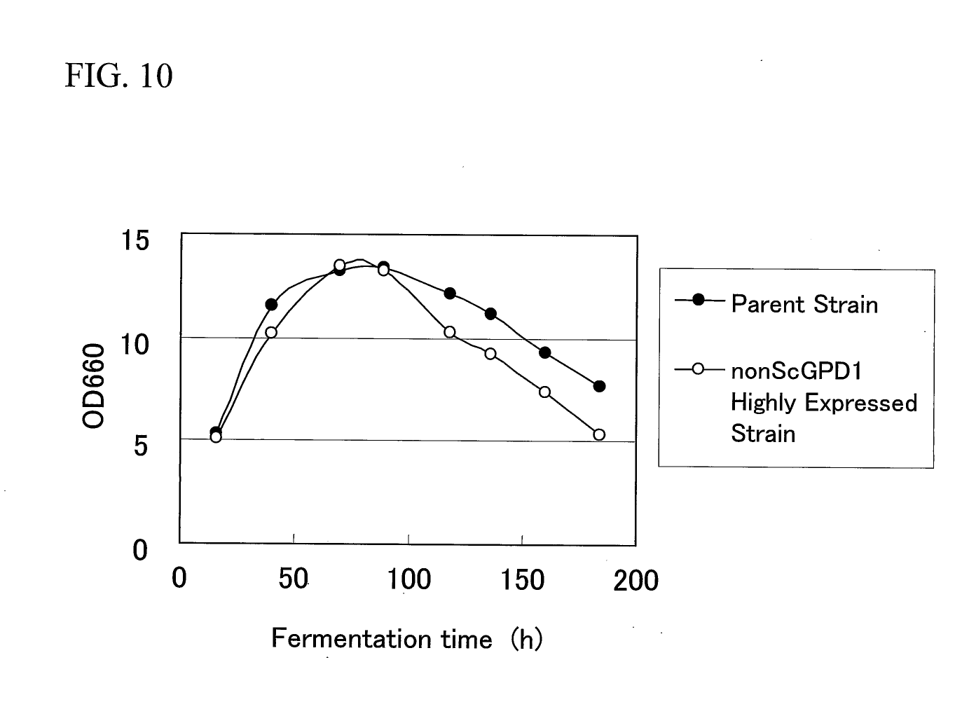

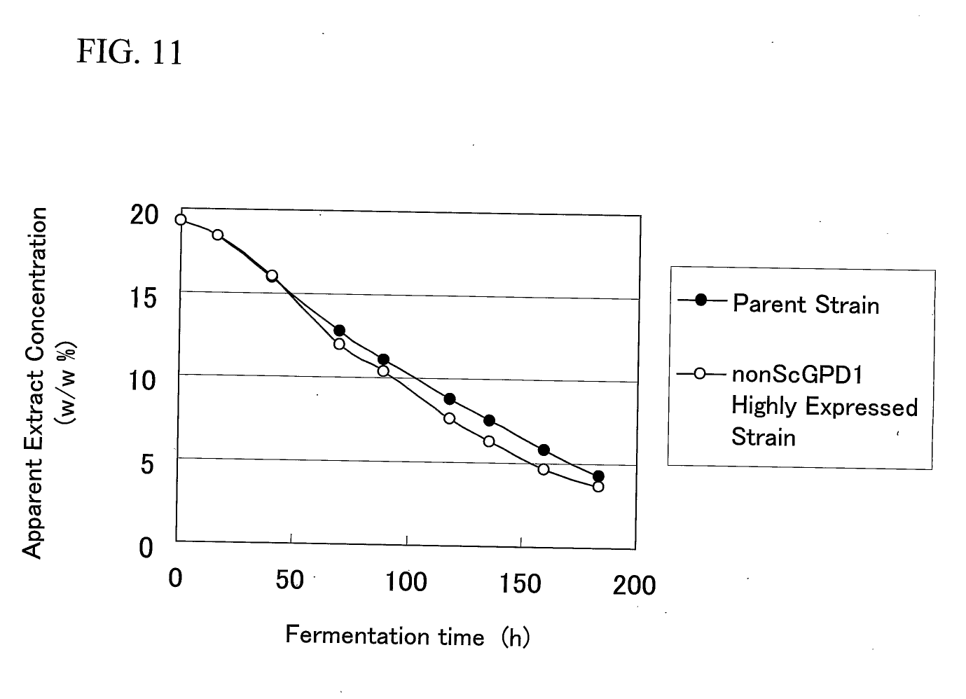

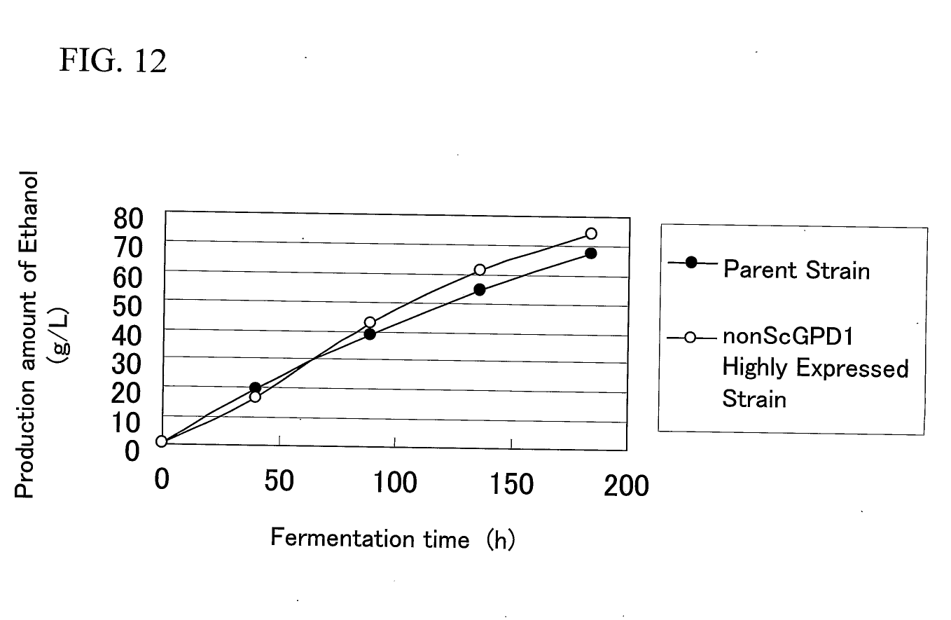

The fermentation broth was sampled over time, and the change over time in the yeast growth rate (OD660) (FIG. 10), the amount of extract consumption (FIG. 11) and the amount of ethanol production (FIG. 12) were determined. Ethanol production amount in the fermentation broth was quantified using F-kit ethanol (product number 176290, manufactured by Roche) (see Z. Anal. Chem. 284: 113-117, 1977). As indicated in FIG. 12, the amount of ethanol production at the completion of fermentation was 68 g4L for the parent strain, and was 74 g/L for non-ScGPD1 highly expressed strain. That is, enhanced fermentation by high expression of non-ScGPD1 was observed.

### Example 8

### Low-Temperature Storage Property Test

Low-temperature storage property test of the parent strain (BH174 strain) and the non-ScGPD1 highly expressed strain obtained by the method described in Example 3 was performed under conditions described below.

Yeast cells cultured on YPD liquid medium (1% yeast extract, 2% polypeptone, 2% glucose) at 30° C. overnight were collected by centrifugation. The collected cells were suspended in 5% ethanol at a density of approximately 80 cells/ml. The suspended cells were kept at 4° C. for 29 days, then viable cell ratio was evaluated by counting dead cells by methylene blue staining (BCOJ Microbiology Method (Biseibutsu Bunsekihou), edited by Brewers Association Japan (Beer Shuzo Kumiai)). Further, the collected cells were suspended similarly in 10% ethanol, and viable cell ratio was evaluated after it was kept at 4° C. for 2 day. As indicated in Table 1, although viable cell ratio of the parent strain in 5% ethanol after 29 days was 45.1%, in 10% ethanol after 2 days was 54.1%, viable cell ratio of the non-ScGPD1 highly expressed strain was 58.7% and 76.6%, respectively. It was demonstrated by the results that viable cell ratio was increased by high expression of non-ScGPD1.

TABLE 1
Viable Cell Ratio After Low-temperature Storage

| strain | suspension | viable cell ratio (%) |
|---|---|---|
| parent strain (BH174 strain) | 5% ethanol | 45.1 |
| non-ScGPD1 highly expressed strain | 5% ethanol | 58.7 |
| parent strain (BH174 strain) | 10% ethanol | 54.1 |
| non-ScGPD1 highly expressed strain | 10% ethanol | 76.6 |

### Example 9

### Frozen Storage Property Test

Frozen storage property test of the parent strain (BH174 strain) and the non-ScGPD1 highly expressed strain obtained by the method described in Example 3 was performed under conditions described below.

Yeast cells cultured on YPD liquid medium (1% yeast extract, 2% polypeptone, 2% glucose) at 30° C. overnight were collected by centrifugation. The collected cells were suspended in water at a density of approximately 80 cells/ml. The suspended cells were kept at −20° C. for 29 days, then viable cell ratio was evaluated by counting dead cells by methylene blue staining (BCOJ Microbiology Method (Biseibutsu Bunsekihou), edited by Brewers Association Japan (Beer Shuzo Kumiai)). As indicated in Table 2, although viable cell ratio of the parent strain was 33.3%, viable cell ratio of the non-ScGPD1 highly expressed strain was 39.3%. It was demonstrated by the results that viable cell ratio was increased by high expression of non-ScGPD1.

TABLE 2
Viable Cell Ratio After Frozen Storage

| strain | viable cell ratio (%) |
|---|---|
| parent strain (BH174 strain) | 33.3 |
| non-ScGPD1 highly expressed strain | 39.3 |

### Example 10

### Drying-Resistant Property Test

Drying-resistant property test of the parent strain (BH1172 strain) and the non-ScGPD1 highly expressed strain obtained by the method described in Example 3 was performed under conditions described below.

Yeast cells cultured on YPD liquid medium (1% yeast extract, 2% polypeptone, 2% glucose) at 30° C. overnight were collected by centrifugation. The collected cells were suspended in water at a density of approximately 80 cells/ml, and then the suspended cells were dried in vacuo. The dried cells were kept at 4° C. for 29 days, and then re-moistened by water. The viable cell ratio was evaluated by counting dead cells by methylene blue staining (BCOJ Microbiology Method (Biseibutsu Bunsekihou), edited by Brewers Association Japan (Beer Shuzo Kumiai)). As indicated in Table 3, although viable cell ratio of the parent strain was 0%, viable cell ratio of the non-ScGPD1 highly expressed strain was 23.3%. It was demonstrated by the results that viable cell ratio was increased by high expression of non-ScGPD1.

TABLE 3
Viable Cell Ratio After Frozen Storage

| strain | viable cell ratio (%) |
|---|---|
| parent strain (BH172 strain) | 0 |
| non-ScGPD1 highly expressed strain | 23.3 |

### Example 11

### Low-Temperature Storage Property Test

Low-temperature storage property test of the parent strain (KN009F strain) and the ScGPD1 highly expressed strain obtained by the method described in FEMS Yeast Res. 2: 225-232 (2002) was performed under conditions described below.

Yeast cells cultured on YPD liquid medium (1% yeast extract, 2% polypeptone, 2% glucose) at 30° C. overnight were collected by centrifugation. The collected cells were suspended in 5% ethanol at a density of approximately 80 cells/ml. The suspended cells were kept at 4° C. for 10 days, then viable cell ratio was evaluated by counting dead cells by methylene blue staining (BCOJ Microbiology Method (Biseibutsu Bunsekihou), edited by Brewers Association Japan (Beer Shuzo Kumiai)). As indicated in Table 4, although viable cell ratio of the parent strain was 64%, viable cell ratio of the ScGPD1 highly expressed strain was 94%. It was demonstrated by the results that viable cell ratio was increased by high expression of ScGPD1.

TABLE 4
Viable Cell Ratio After Low-temperature Storage

| strain | viable cell ratio (%) |
|---|---|
| parent strain (KN009F strain) | 64 |
| ScGPD1 highly expressed strain | 94 |

### INDUSTRIAL APPLICABILITY

According to the method for producing alcoholic beverages, alcoholic beverages with superior flavor can be produced because the amount of glycerol, which provides body and mellowness to the product, is enhanced. Further, a yeast with a superior low-temperature storage property, frozen storage property or drying resistant property can be provided by the present invention. Moreover, fermentation time can be reduced in high concentration brewing by using the brewery yeast of the present invention.

1. A polynucleotide selected from the group consisting of:
(a) a polynucleotide comprising a polynucleotide consisting of the nucleotide sequence of SEQ ID NO: 1 or SEQ ID NO: 3;
(b) a polynucleotide comprising a polynucleotide encoding a protein consisting of the amino acid sequence of SEQ ID NO: 2 or SEQ ID NO: 4;
(c) a polynucleotide comprising a polynucleotide encoding a protein consisting of the amino acid sequence of SEQ ID NO: 2 or SEQ ID NO: 4 in which one or more amino acids thereof are deleted, substituted, inserted and/or added, and having a glycerol-3-phosphate dehydrogenase activity;
(d) a polynucleotide comprising a polynucleotide encoding a protein having an amino acid sequence having 60% or higher identity with the amino acid sequence of SEQ ID NO: 2 or SEQ ID NO: 4, and said protein having a glycerol-3-phosphate dehydrogenase activity;
(e) a polynucleotide comprising a polynucleotide which hybridizes to a polynucleotide consisting of a nucleotide sequence complementary to the nucleotide sequence of SEQ ID NO: 1 or SEQ ID NO: 3 under stringent conditions, and which encodes a protein having a glycerol-3-phosphate dehydrogenase activity; and
(f) a polynucleotide comprising a polynucleotide which hybridizes to a polynucleotide consisting of a nucleotide sequence complementary to the nucleotide sequence of the polynucleotide encoding the protein having the amino acid sequence of SEQ ID NO: 2 or SEQ ID NO: 4 under stringent conditions, and which encodes a protein having a glycerol-3-phosphate dehydrogenase activity.

2. The polynucleotide according to claim 1 selected from the group consisting of:
(g) a polynucleotide comprising a polynucleotide encoding a protein consisting of the amino acid sequence of SEQ ID NO: 2 or SEQ ID NO: 4, or encoding the amino acid sequence of SEQ ID NO: 2 or SEQ ID NO: 4 in which 1 to 10 amino acids thereof are deleted, substituted, inserted, and/or added, and wherein said protein has a glycerol-3-phosphate dehydrogenase activity;
(h) a polynucleotide comprising a polynucleotide encoding a protein having 90% or higher identity with the amino acid sequence of SEQ ID NO: 2 or SEQ ID NO: 4, and having a glycerol-3-phosphate dehydrogenase activity; and
(i) a polynucleotide comprising a polynucleotide which hybridizes to a polynucleotide consisting of a nucleotide sequence of SEQ ID NO: 1 or SEQ ID NO: 3 or which hybridizes to a polynucleotide consisting of a nucleotide sequence complementary to the nucleotide sequence of SEQ ID NO: 1 or SEQ ID NO: 3, under high stringent conditions, which encodes a protein having a glycerol-3-phosphate dehydrogenase activity.

3. The polynucleotide according to claim 1 comprising a polynucleotide consisting of the nucleotide sequence of SEQ ID NO: 1 or SEQ ID NO: 3.

4. The polynucleotide according to claim 1 comprising a polynucleotide encoding a protein consisting of the amino acid sequence of SEQ ID NO: 2 or SEQ ID NO: 4.

5. The polynucleotide according to claim 1, wherein the polynucleotide is DNA.

6. A protein encoded by the polynucleotide according to claim 1.

7. A vector containing the polynucleotide according to claim 1.

8. A yeast into which the vector according to claim 7 has been introduced.

9. The yeast according to claim 8, wherein glycerol producing ability is increased.

10. The yeast according to claim 8, wherein low-temperature storage property, frozen storage property or drying-resistant property is increased.

11. The yeast according to claim 8, wherein osmotic pressure resistant property is increased.

12. The yeast according to claim 9, wherein the glycerol producing ability is increased by increasing an expression level of the protein encoded by the polynucleotide.

13. The yeast according to claim 10, wherein the low-temperature storage property, frozen storage property or drying-resistant property is increased by increasing an expression level of the protein encoded by the polynucleotide.

14. The yeast according to claim 11, wherein the osmotic pressure resistant property is increased by increasing an expression level of the protein encoded by the polynucleotide.

15. A method for producing an alcoholic beverage by using the yeast according to claim 8.

16. The method according to claim 15, wherein the brewed alcoholic beverage is a malt beverage.

17. The method according to claim 15, wherein the brewed alcoholic beverage is wine.

18. An alcoholic beverage produced by the method according to claim 15.

19. A method for assessing a test yeast for its glycerol producing ability, comprising using a primer or probe designed based on the nucleotide sequence of a glycerol-3-phosphate dehydrogenase gene having the nucleotide sequence of SEQ ID NO: 1 or SEQ ID NO: 3.

20. A method for assessing a test yeast for its glycerol producing ability, comprising: culturing the test yeast; and measuring the expression level of the glycerol-3-phosphate dehydrogenase gene having the nucleotide sequence of SEQ ID NO: 1 or SEQ ID NO: 3.

21. A method for selecting a yeast, comprising: culturing test yeasts; quantifying the protein of claim 6 or measuring the expression level of the glycerol-3-phosphate dehydrogenase gene having the nucleotide sequence of SEQ ID NO: 1 or SEQ ID NO: 3; and selecting a test yeast having an amount of the protein or the gene expression level according to desired glycerol producing ability.

22. The method for selecting a yeast according to claim 21, comprising: culturing a reference yeast and test yeasts; measuring for each yeast the expression level of the glycerol-3-phosphate dehydrogenase gene having the nucleotide sequence of SEQ ID NO: 1 or SEQ ID NO: 3; and selecting a test yeast having the gene expression higher than that in the reference yeast.

23. The method for selecting a yeast according to claim 21, comprising: culturing a reference yeast and test yeasts; quantifying the protein encoded by the polynucleotide in each yeast; and selecting a test yeast having a larger amount of the protein than that in the reference yeast.

24. A method for producing an alcoholic beverage comprising: conducting fermentation using the yeast according to claim 8 or a yeast selected by the methods according to claim 21.

25. A method for enhancing low-temperature storage property of yeast by using a vector comprising a polynucleotide selected from the group consisting of:
(j) a polynucleotide comprising a polynucleotide encoding a protein consisting of the amino acid sequence of SEQ ID NO: 10, or encoding the amino acid sequence of SEQ ID NO: 10 in which 1 to 10 amino acids thereof are deleted, substituted, inserted, and/or added, and wherein said protein has a glycerol-3-phosphate dehydrogenase activity;
(k) a polynucleotide comprising a polynucleotide encoding a protein having 90% or higher identity with the amino acid sequence of SEQ ID NO: 10, and having a glycerol-3-phosphate dehydrogenase activity; and
(l) a polynucleotide comprising a polynucleotide which hybridizes to a polynucleotide consisting of a nucleotide sequence of SEQ ID NO: 9 or which hybridizes to a polynucleotide consisting of a nucleotide sequence complementary to the nucleotide sequence of SEQ ID NO: 9, under high stringent conditions, which encodes a protein having a glycerol-3-phosphate dehydrogenase activity.
